(19) United States
(12) Patent Application Publication
(10) Pub. No.: US 2016/0106789 A1
(43) Pub. Date: Apr. 21, 2016

(54) Inhibition of Neurodegenerative Disease by Grape Seed Extract, Green Tea Extract and Probiotic Bacteria

(71) Applicants: Gil NOFAR, Haifa (IL); Uri COGAN, Haifa (IL)

(72) Inventors: Gil NOFAR, Haifa (IL); Uri COGAN, Haifa (IL)

(21) Appl. No.: 14/978,859

(22) Filed: Dec. 22, 2015

Related U.S. Application Data
(63) Continuation-in-part of application No. 14/776,170, filed on Sep. 14, 2015.
(60) Provisional application No. 61/781,761, filed on Mar. 14, 2013.

(51) Int. Cl. A61K 35/747; A61K 36/87; A61K 36/82; A61K 9/48

(52) U.S. Cl. CPC A61K 35/747; A61K 9/4891; A61K 36/87; A61K 36/82; A61K 2035/115

(57) ABSTRACT

A composition is provided comprising extracts of at least grape seeds (GSE) and green tea. The extracts are preferably polyphenolic and preferably comprise probiotic bacteria. The use of extracts from green tea and grape seeds for the manufacture of a medicament against a neurodegenerative disease is also disclosed.

### CLAIM OF PRIORITY

This application claims priority to U.S. application Ser. No. 14/776,170 filed on Sep. 14, 2015 which is a national stage entry of International Application PCT/IL2014/050265 filed on Mar. 13, 2014 which claims priority to U.S. Application 61/781,761 filed on Mar. 14, 2013, the contents of which are herein fully incorporated by reference in its entirety.

### FIELD OF THE INVENTION

The present invention relates to the treatment, medicaments and use of particular compositions for manufacture thereof, for use against the progression of, or regression from dementia and neurodegenerative diseases such as Alzheimer disease.

### BACKGROUND OF THE INVENTION

Alzheimer's disease (AD), is a major form of dementia that affects memory, thinking, and behavior, and gradually worsens over time.

There are two types of AD:
Early onset AD: Symptoms appear before age 60. This type is much less common than late onset. However, it tends to rapidly worsen. Early onset disease can run in families. Several genes have been identified with susceptibility to the syndrome.
Late onset AD: This is the most common type. It occurs in people age 60 and older. It may run in some families, but the role of genes in the occurrence of late AD is less clear.

The cause of late AD is not clear. Our genes and environmental factors seem to play a role. See for example: http://www.ncbi.nlm.nih.gov/pubmedhealth/PMH0001767 (1).

Epigallocatechin-3-gallate (EGCG) the main polyphenolic constituent of green tea, has been shown in numerous studies to be a highly effective antioxidant, by reacting with the majority of reactive oxygen species (ROS) (2).

It was also suggested that in addition to its radical scavenging, chelation of redox active metals such as iron, and regulation of antioxidant protective enzymes, can be part of the protective effect of EGCG against neuronal diseases (3). Indeed, the chemical pathologies of AD and of Parkinson disease (PD) show many similarities, including increased iron concentration in addition to oxidative stress (3).

Experimental work with green tea and EGCG also indicates that orally ingested polyphenols can reach the brain, and exert neuroprotective effects (3). In fact, in-vitro studies showed that EGCG prevented the formation of β-sheet rich aggregation products, such as amyloid fibrils, that are associated with the development of AD and PD, leading the authors to conclude that EGCG could be useful for therapy against development of these diseases (4).

In a different study it was shown that EGCG reduces the generation of β-amyloid peptides, raising the possibility that EGCG dietary supplementation may provide effective prophylaxis for AD (5). Furthermore, in a study which examined the association between green tea consumption and cognitive function, it was shown that higher consumption of green tea is associated with lower prevalence of cognitive impairment in humans, supporting the potential benefit of green tea and EGCG in preventing neurodegenerative diseases (6).

The grape seed extract (GSE) is a water extract of grape seeds, for example Meganatural-BP® produced by Polyphenolics Inc. (7). The extract is comprised of polyphenol monomers, oligomers and polymers of catechin and of epicatechin (8, 9). EGCG and the polyphenols in GSE belong to a similar family of polyphenols, yet they are chemically distinct. Consumption of this particular GSE (produced by Polyphenolics Inc.) in a clinical study was found to reduce both the systolic and diastolic blood pressures (10).

A different study demonstrated that the same GSE product prevented amyloid β-protein aggregation into high molecular weight (HMW) oligomers, in-vitro, and when administrated orally to mice that serve as a model for AD, the GSE attenuated AD type cognitive deterioration, coincidentally with reduced HMW (High Molecular Weight) soluble oligomeric amyloid β in the brain (11).

A recent study (12) shows that certain strains of lactic acid bacteria, when consumed orally as probiotics, like the specific strain Lactobacillus plantarum, possess enzymes that can effectively breakdown polyphenols and tannins into smaller metabolites, that are more easily absorbed from the intestine into the blood stream. Such bacteria need to be protected from gastric conditions.

It should be further noted that capsules named “DRCAPST™ capsules”, a product of “Capsugel”, are used to house and protect the active ingredients (13), and consist of the acid resistant polymer, hydroxypropyl methylcellulose. In vitro studies showed that the capsule's contents are protected for at least 20 min under gastric conditions and are fully released at an intestinal pH of 6.8 (13). This delayed release essentially protects the capsule's ingredients from the harsh environment in the stomach.

### SUMMARY OF THE INVENTION

According to one aspect, a composition comprising extracts of grape seeds (GSE) and green tea, and probiotic bacteria is provided.

In preferred embodiments the extracts are polyphenolic.

In some preferred embodiments, the green tea extract is primarily EGCG.

According to another aspect, a composition comprising polyphenol extracts from at least two plants, and probiotic bacteria is provided.

The extracts are for example from plants selected from a group consisting of: grapes, tea, pomegranate, dates, and berries.

The probiotic bacteria are for example selected from Lactobacilus plantarum, bifidobacteria, and mixtures thereof.

The bifidobacteria may be selected from Bifidobacterium pseudocatenulatum B7003 and Bifidobacterium animalis ssp. lactis LAFTI@B94, and mixtures thereof

According to yet another aspect, capsules comprising: EGCG polyphenols; probacteria selected from L. plantarum, probiotic bacteria, and mixtures thereof, and polyphenol extract from GSE are provided.

In some embodiments, the capsules are HPMC capsules.

In some preferred embodiments the capsules consist of: EGCG polyphenols; probacteria selected from L. plantarum, probiotic bacteria, and mixtures thereof, and polyphenol extract from GSE.

In some embodiments the polyphenols and the probacteria are segregated from each other.

Preferably, the GSE is at least 100 mg.

Preferably, the amount of EGCG is at least 50 mg.

According to one aspect, treatment of a neurodegenerative disease is provided, the treatment consisting of administering to a patient a composition comprising probiotic bacteria and polyphenolic extracts from green tea and grape seeds.

The neurodegenerative disease may be one or more of the diseases: Alzheimer's, Parkinson's, ALS and Huntington's.

The composition may consist of probiotic bacteria and polyphenolic extracts from green tea and grape seeds.

The composition may consist of a capsule comprising probiotic bacteria and polyphenolic extracts from green tea and grape seeds. According to another aspect, improving cognition in a patient is provided, the improving consisting of administering to the patient a composition comprising probiotic bacteria, and extracts from green tea and grape seeds.

According to yet another aspect, manufacture of Grape seed extract is provided, the manufacture comprising:

contacting a member selected from the group consisting of whole grapes, grape seeds, grape pomace, and mixtures thereof with water at an elevated temperature to obtain a crude grape-water extract;

treating the crude grape-water extract with a tannase enzyme at an elevated temperature to obtain a polyphenol grape extract;

acidifying the polyphenol grape extract to a pH of about 1.5 to 2.5 to obtain an acidified polyphenol extract;

cooling the acidified polyphenol extract; and

filtering the cooled acidified polyphenol extract to obtain a filtered polyphenol extract, wherein the polyphenols consist of low molecular weight polyphenols, and wherein the Grape seeds are unfermented.

Preferably, the Grape seed extract comprises between 5-15% monomers, 5-20% dimers, 3-10% trimers, 2-10% tetramers, and 2-10% pentamers by weight, and having 2% by weight or less epicatechin-gallate terminal units.

### DESCRIPTION OF THE PREFERRED EMBODIMENTS

Before explaining at least one embodiment of the invention in detail, it is to be understood that the invention is not necessarily limited in its application to the details set forth in the following description. The invention is capable of other embodiments or of being practiced or carried out in various ways.

The terms “comprises”, “comprising”, “includes”, “including”, and “having” together with their conjugates mean “including but not limited to”.

The term “consisting of” has the same meaning as “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials and methods are illustrative only and not intended to be limiting.

No attempt is made to show structural details of the invention in more detail than is necessary for a fundamental understanding of the invention, the description making apparent to those skilled in the art how the several forms of the invention may be embodied in practice.

Preparations and applications of medicaments comprising at least two of EGCG, GSE and L. plantarum are provided, that, surprisingly synergistically improve mental abilities such as memory and are thus thought to also inhibit or at least reduce the progression of diseases such as Alzheimer disease or PD. Preferably, the medicament comprises at least EGCG and GSE.

### Methods and Materials

Capsules

Meganatural-BP® is 300 mg GSE provided in HPMC (hydroxypropylmethyl cellulose) capsules (DR caps®) of Capsugel, which are gastric-resistant and mostly disintegrate in the small intestine.

EGCG (Lyan, China) and Lactobacillus plantarum R1012-150 (Institut Rosell, Canada) DR Caps capsules were prepared. The average amount of Lactobacillus plantarum per capsule was 377 mg/capsule (about 6*10¹⁰ bacteria). The bacteria should be kept in cool conditions—about 5° C. The average amount of EGCG was 218 mg. The EGCG is produced by water/EtOH extraction from leaves of locally cultivated camellia sinensis O. Ktze.

The catechins content in the EGCG is preferably at least 95%. The content of polyphenols in GSE is preferably at least 90%.

Green tea and grape seed extracts as well as bacteria from other suppliers are considered to have an effect similar to that from these material sources.

The minimum effective dosage of GSE per day is considered to be about 100 mg, and the minimum effective dosage of EGCG is about 50 mg per day; the dosage depends upon the severity of the condition and the patient's general state of health, as well as weight and other considerations familiar to the practitioner.

In other embodiments the medicament comprises other probiotic bacteria, in addition to, or instead of, Lactobacillus plantarum.

The probiotic bacteria, Bifidobacterium pseudocatenulatum B7003, a strain of Bifidobacteria, was found to effectively convert by fermentation the flavonoids of legume milks, such as that of soybeans and of other bean derived milks into their respective aglycon forms. In addition the B7003 strain possesses the capability of adhering to gut epithelial cells [Flavonoid bioconversion in Bifidobacterium pseudocatenulatum B7003: A potential probiotic strain for functional food development; Di Gioia, et al., Journal of Functional Foods (2014), 7, 671-679]

The probiotic bacteria Bifidobacterium animalis ssp. lactis LAFTI@B94 was found to utilize green tea polyphenols, such as epigallocatechin, catechin and epicatechin, as the sole source of carbon. Biotransforming the flavonoids by the probiotic bacteria resulted in increase of the biological activities of the modified flavonoids, such as their antioxidant properties, and ACE (Angiotensin-Converting Enzyme) inhibition capacity. The study led to the conclusion that incorporation of B. animalis B94 in various polyphenol-rich foods could be a way of improving the antioxidant properties of the foods, and that polyphenol-rich food could act as a matrix for probiotic strains.

Reference: Biotransformation and resulting biological properties of green tea polyphenols produced by probiotic bacteria.

Ana M. López de Lacey, Efrén Pérez-Santín, M. Elvira López-Caballero*, Pilar Montero. LWT—Food Science and Technology 58 (2014) 633-638

### EXAMPLES

It is notable that although full clinical trials involve control by placebo, the preliminary results from treatment of a patient suffering from AD, described below, are absent this control. However, such control is perhaps of little relevance since, to the best of the inventors' knowledge no reliable and perceptible change would be observed in administration of placebos to such patients having cognitive impairment.

Since the polyphenols generally react with proteins, the administration is best performed on an empty stomach.

General Effect of GSE on Mental Faculties

A first subject consumed a capsule containing 300 mg of Meganatural-BP® over a period of 18 months, once daily, immediately after nocturnal sleep. Some improvement of memory was observed during the period that the GSE was administered, compared to memory ability before the administration commenced.

General Effect of EGCG on Mental Faculties

The same subject ceased to consume GSE and immediately subsequent consumed capsules of 100 mg EGCG (Lyan) over a period of 40 days, once daily, immediately after nocturnal sleep.

No improvement of memory was observed during the period that the EGCG was administered.

General Effect of Combined GSE and EGCG on Mental Faculties

300 mg of Meganatural-BP® and 60 mg of EGCG capsules were concomitantly administered to the same subject, immediately after nocturnal sleep, following the EGCG regime.

There was a pronounced improvement of long-term memory which had not been previously observed by an observation group as well as by the subject. The improvements were both in the level of detail and in the completeness of the memory. For example, location/people details of a family trip were recalled that no other members of the family nor indeed the subject himself could previously recall; details of an artwork observed over 40 years previously by the subject and a group of people were recalled in minute details, including the location of the creation, shape and its colors, whereas none of the members of the group could recall any of these details.

The subject also reported feeling generally physically well and more lucid in mind.

It is noteworthy that three months into the combined GSE and EGCG regime, the subject was found to have below normal levels of folic acid. It is known that there is a positive correlation between folic acid and memory, thus the apparent improvement in memory may be despite folic acid deficiency.

Effect of Combined GSE and EGCG on Alzheimer Patients A hypertensive and diabetic 82 year old male exhibited a cognitive deterioration and subsequently (after about a year) underwent mini-mental state examination (MMSE) which produced a grade of 13; thus the patient was diagnosed as suffering from Alzheimer disease.

Six weeks after the diagnosis, the patient's condition continued to deteriorate. The patient was administered memantine hydrochloride (Ebixa® tablet), an antidementia medicine, on a daily basis. Blood-pressure reducing medication was concomitantly administered and the blood pressure was measured to be on the average of 130/70 systolic/diastolic. However, the patient's deterioration appeared to be unaffected by the drug and administration of Ebixa was consequently discontinued after two weeks.

The following four months were characterized by sporadic memory lapses typical of a moderately severe affliction as well as behavioral deteriorations that occurred from time to time

At this point of time, according to a 60 days plan, the patient was daily administered two Meganatural-BP® capsules (600 mg), one 218 mg EGCG capsule (Lyan) and one L. plantarum capsule (377 mg), as well as blood-pressure reducing medication.

About 18 days after the administration (of the GSE. EGCG, and bacteria) was initiated, the blood pressure dropped to about 70-80/50.

Consequently, the blood-pressure reducing medication was discontinued and the administration of GSE was reduced to one capsule per day (300 mg) as GSE is known to reduce blood pressure.

Two weeks (14 days) thereafter the reduction of blood pressure, the deterioration appeared to have halted.

During the next two weeks, a gradual improvement of the mental capacities such as memory-related performance was observed.

In the next two weeks, a significant improvement in the cognitive state of the patient was observed and his mental state started to become more stable. Regression of the disease was noted.

At this point, the GSE, EGCG, and bacteria were discontinued and the patient's mental capacities appeared to have dramatically improved compared to his condition just prior to the administration of the GSE, EGCG and probiotic bacteria, in particular his ability to follow a clock and sense time appeared to have been restored and there were no memory lapses.

No adverse effects on blood sugar were observed during and after the treatment.

Slight memory lapses were observed subsequent to the discontinuation, and a repeat MMSE performed 3 weeks later gave a grade of 19, which is still a highly significant improvement over the patient's performance in the MMSE prior to the administration of GSE, EGCG, and bacteria.

It should be mentioned that Cipralex®, a drug against depression and anxiety, as well as other calming drugs were administrated to the patient prior and during the administration of the GSE, EGCG, and bacteria according to need.

The GSE may be the extract described in any of the patents U.S. Pat. Nos. 7,767,235, 7,651,707 and 6,544,581, and may be prepared according to the process described in U.S. Pat. No. 8,075,929; however, other similar preparations and processes may be investigated whether they contribute to a similar synergistic effect, for example more particular ingredients of the GSE may be used.

The capsules may comprise both the bacteria and the GSE and/or EGCG; having all the components together may simplify the medication regime; however, it may be that the GSE and/or EGCG reduce the effectiveness of the medication, presumably due to an adverse interaction of substances in the GSE/EGCG with the bacteria. Therefore, in some embodiments the bacteria are separately provided in gastric-resistant capsules or other dosage forms.

In other embodiments the bacteria and/or GSE/EGCG extracts (either or both) are enteric coated, and are provided as enteric-coated granules within one dose, e.g. capsule. Presumably the bacteria perform most of their beneficial activity in the large intestine; and most of the bacteria are likely to arrive there intact when protected from the gastric conditions in the stomach.

Alternatively, the bacteria and the polyphenols may be provided in separate compartments. Such segmented capsules/pills may be manufactured by 3-D printing for example, with the materials therein, or added after the pill/capsule is ready.

Other forms of delivery may be used instead of or in addition to capsules, including but not limited to nutraceuticals, tablets, suspensions, suppositories (liquid or solid), injectable/intravenous solutions and lotions. The solid forms of delivery may be at least partially 3-D printed.

Some embodiments further comprise flavourings and/or food stuff, to help consume the materials. The foodstuff is preferably low-protein.

In some embodiments the bacteria are physically separated or functionally isolated from the EGCG and GSE extracts until they all arrive at the intestines; or at least they remain as separate as possible until arrival at the large intestine. Polysaccharide coating of the GSE, EGCG and bacteria, or at least the bacteria, may help delay the interaction until the optimal time.

Surprising synergy of the (presumably polyphenolic) ingredients in grape seed and green tea extracts has been demonstrated for improvement of mental condition or at least helping to reduce mental deterioration, in particular when properly used in conjuction with probiotic bacteria that are thought (without being bound to the theory) to provide metabolic products with improved efficacy. Other synergies that are considered likeliest are between the GSE and/or green-tea EGCG and polyphenolic extracts from other plant sources, in particular from pomegranates, dates, and various berries or vitamin E. other oxidants or polyphenols can be used without limiting the scope of the invention.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### CITED LITERATURE

1) http://www.ncbi.nlm.nih.gov/pubmedhealth/PMH00017672. Yang, C. S. et al.
2) Annual Review of Pharmacology and Toxicology 42, 25-54 (2002).
3) Weinreb O. et al. J. Nutritional Biochemistry 15 (9) 506-516) (2004)
4) Ehrnhoefer, D. E. et al. Nature Structural & Molecular Biology 15 (6) 558-566 (2008).
5) Rezai-Zadeh, K. et al. J. of Neoroscience 25, 8807-8814 (2005).
6) Kuriyama S. et al. Am. J. Clinical Nutrition 83 (2), 355-361 (2006).
7) U.S. Pat. No. 6,544,581 (2003).
8) U.S. Pat. No. 7,767,235 (2010).
9) A Brochure on MegaNatural-BP a patented Grape Seed Extract. Published by Polyphenolics, P.O. Box 99, Madera Calif. 93639 (www.Polyphenolics.com)
10) Sivapraksapillai, B. et al. Metabolism Clinical and Experimental 58, 1743-1746 (2009).
11) Wang, J. et al. The Journal of Neuroscience 28 (25), 6388-6392, (2008).
12) Jakesevic, M., Doctoral Thesis, Lund University, Sweden (2011).
13) DRcaps™ capsules. www.capsugel.com

1. A composition comprising extracts of grape seeds (GSE), green tea, and probiotic bacteria.

2. The composition of claim 1, wherein the extracts are polyphenolic.

3. The composition of claim 1, wherein the green tea extract is primarily EGCG.

4. A composition comprising polyphenol extracts from at least two plants, and probiotic bacteria.

5. The composition of claim 4, wherein the extracts are from plants selected from a group consisting of: grapes, tea, pomegranate, dates, and berries.

6. The composition of claim 1, wherein the probiotic bacteria is selected from Lactobacilus plantarum, bifidobacteria, and mixtures thereof.

7. The composition of claim 6, wherein the bifidobacteria is selected from Bifidobacterium pseudocatenulatum B7003 and Bifidobacterium animalis ssp. lactis LAFTI@B94, and mixtures thereof.

8. Capsules comprising: EGCG polyphenols; probiotic bacteria selected from L. plantarum, bifidobacteria, and mixtures thereof, and polyphenol extract from GSE.

9. The capsules of claim 8, wherein the capsules are gastric resistant.

10. The capsules of claim 9, wherein the capsules comprise HPMC The capsules of claim 8, wherein the probacteria are enteric coated.

11. The capsules of claim 8, wherein the polyphenols and the probiotic bacteria are segregated from each other.

12. The capsules of claim 8, wherein the GSE is at least 100 mg.

13. The capsules of claim 8, wherein the amount of EGCG is at least 50 mg.

14. Treatment of a neurodegenerative disease consisting of administering to a patient a composition comprising probiotic bacteria and polyphenolic extracts from green tea and grape seeds.

15. The treatment of claim 14, wherein the neurodegenerative disease is selected from one or more of the diseases: Alzheimer's, Parkinson's, ALS and Huntington's.

16. Improving cognition in a patient consisting of administering to the patient a composition comprising probiotic bacteria, and extracts from green tea and grape seeds.

17. The improving cognition of claim 16, wherein the extracts are polyphenolic.

18. Manufacture of grape seed extract comprising:
contacting a member selected from the group consisting of whole grapes, grape seeds, grape pomace, and mixtures thereof with water at an elevated temperature to obtain a crude grape-water extract;
treating the crude grape-water extract with a tannase enzyme at an elevated temperature to obtain a polyphenol grape extract;
acidifying the polyphenol grape extract to a pH of about 1.5 to 2.5 to obtain an acidified polyphenol extract;
cooling the acidified polyphenol extract, and
filtering the cooled acidified polyphenol extract to obtain a filtered polyphenol extract, wherein the polyphenols consist of low molecular weight polyphenols, and wherein the Grape seeds are unfermented.

19. The manufacture of claim 18, wherein the Grape seed extract comprises between 5-15% monomers, 5-20% dimers, 3-10% trimers, 2-10% tetramers, and 2-10% pentamers by weight, and having 2% by weight or less epicatechin-gallate terminal units.
